(19) United States
(12) Patent Application Publication
(10) Pub. No.: US 2018/0028767 A1
(43) Pub. Date: Feb. 1, 2018

(54) TOPICAL SPRAY SYSTEM OF HALOBETASOL

(71) Applicant: SUN PHARMACEUTICAL INDUSTRIES LIMITED, Mumbai (IN)

(72) Inventors: Anil RANA, Chandigarh (IN); Lav KESHRI, Lucknow (IN); Simrata BEDI, Gurgaon (IN); Ravi KOCHHAR, Gurgaon (IN)

(21) Appl. No.: 15/656,806

(22) Filed: Jul. 21, 2017

(30) Foreign Application Priority Data
Jul. 27, 2016 (IN) 201611025670
Jun. 9, 2017 (IN) 201711020238

(51) Int. Cl. A61M 11/04; A61M 39/22; A61K 9/12; A61M 35/00; A61K 31/573; A61K 9/00

(52) U.S. Cl. CPC A61M 11/04; A61K 31/573; A61K 9/0014; A61K 9/12; A61M 35/003; A61M 39/22; F16K 1/12

(57) ABSTRACT

The present invention relates to a halobetasol topical spray system comprising: (i) a composition comprising halobetasol; (ii) a dispensing system comprising: (a) a pressurized container comprising the composition and (b) a metered valve assembly comprising a metered valve having a stem connected to an actuator, wherein the actuator is provided with an insert having an orifice diameter in a range of about 0.2 mm to about 1.5 mm, wherein the actuator is depressed for activation to expel the composition from the pressurized container forming a focused coverage area. Further, said compositions are delivered from a dispensing system in metered doses.

### RELATED APPLICATIONS

This application claims priority to IN201611025670, filed Jul. 27, 2016 and IN201711020238, filed Jun. 9, 2017, both of which are hereby incorporated by reference in their entireties.

### FIELD OF THE INVENTION

The present invention relates to a topical spray system of halobetasol comprising: (i) a composition comprising halobetasol; (ii) a dispensing system comprising: (a) a pressurized container comprising the composition and (b) a metered valve assembly comprising a metered valve having a stem connected to an actuator, wherein the actuator is provided with an insert having an orifice diameter in a range of about 0.2 mm to about 1.5 mm, wherein the actuator is depressed for activation to expel the composition from the pressurized container forming a focused coverage area. Further, said compositions are delivered from the dispensing system in metered doses.

### BACKGROUND OF THE INVENTION

Halobetasol is a super potent corticosteroid widely used for the treatment of dermal ailments. Topical dosage forms of halobetasol such as cream, ointment, and lotion are commercially available under the trade name Ultravate® and have been used for the relief of the inflammatory and pruritic manifestations of corticosteroid responsive dermatoses.

The available cream, ointment, and lotion dosage forms of halobetasol pose high level of patient non-compliance. The physical application of these dosage forms is not desirable where touching of sore or otherwise adversely affected skin need to be minimized in order to avoid any additional discomfort. Further, high viscosity of these dosage forms makes physical application difficult when intended to apply on large areas of the skin. Also, the available dosage forms are unable to deliver the metered dose of the composition which is highly desirable for a super-potent corticosteroid to avoid any unnecessary adverse side effects. Additionally, the available dosage forms remains prone to contamination during use.

Pharmaceutical spray provides the best alternative and exhibits numerous advantages over existing dosage forms of halobetasol. These advantages include ease of application, the possibility of controlling the dose, and the absence of contamination during use. However, there remains some of the problems associated with the spray dosage forms. Clobetasol, another super potent corticosteroid is available in the spray dosage form under the trade name Clobex®. Clobex® nevertheless forms a wide coverage area upon its application for treating skin disorders such as psoriasis. Large coverage area of the skin than the intended coverage area causes unnecessary exposure of the super potent corticosteroid to the body thus leading to systemic side effects. Some of the spray may also escape into the environment when applied to narrow body parts leading to wastage of the composition as well as causing unnecessary exposure to any person present in the vicinity of the patient. Coverage area thus plays an important role in case of medicated sprays especially for a super-potent corticosteroid where unnecessary exposure is highly undesirable. Therefore, in order to make the effective usage of a spray, there remains a need to have an optimum coverage area.

There is an unmet need in the art to develop a topical spray of halobetasol which forms a narrow and focused coverage area, upon its application onto the desired site of action, avoiding any unnecessary exposure of the drug to the body. Also, there remains a need in the art to deliver a metered dose of halobetasol to the desired site of action.

The scientists of the present invention have developed a topical spray of halobetasol which when delivered through a dispensing system exhibits a focused coverage area. The coverage area is determined by the characteristics of the plume geometry and spray pattern. Also, said compositions are delivered from a dispensing system in metered doses which is a significant advance over available cream, ointment, and lotion dosage forms of halobetasol. The particular design of the dispensing system with specific dimensions of an actuator and an insert determine the spray pattern characteristics such that the spray on expulsion forms a focused coverage area. Further, the compositions can be delivered to various body parts including the narrow body parts or body parts having a hairy skin without any wastage of the composition, thus ensuring the safe and effective use. The present compositions also provide the additional benefit of avoiding any physical contact except by the spray itself and thus offer enhanced patient compliance. Further, the inventors have surprisingly found that the spray of present invention shows enhanced permeation into the skin layers as compared to the marketed halobetasol cream formulation.

### SUMMARY OF THE INVENTION

The topical halobetasol spray of the present invention provides focused coverage area, ease of application, and enhanced level of patient compliance. The coverage area of the present invention is determined by the characteristics of the plume geometry e.g. plume angle, plume width, and spray pattern. The focused coverage area helps to avoid the unnecessary exposure of the drug to the body which is highly desirable for a super potent corticosteroid. The focused coverage area thus ensures safe and effective use of the composition. Also, composition of the present invention is delivered in metered doses which prevents both overdosing and under-dosing. The present invention further provides touch-free, easy to use, preservative-free, storage-stable topical spray composition of halobetasol with enhanced patient compliance. Evaporation of the propellant provides a cooling effect which is well received by patients. The composition provides ease of application in difficult to reach areas such as scalp. The halobetasol spray composition of the present invention comprises halobetasol, a non-aqueous solvent, and a propellant. It also relates to processes for the preparation of the topical spray compositions. It further relates to a method of treating skin disorders including corticosteroid responsive dermatoses by administering said topical spray compositions.

### BRIEF DESCRIPTION OF THE DRAWINGS

FIG. 1: Schematic diagram of a partial cut-away view of a dispensing system of the present invention

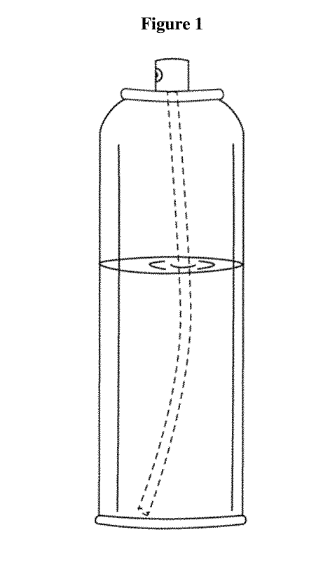

FIG. 2: Schematic diagram of the metered valve of a dispensing system of the present invention

FIG. 3: Plume of (a) Halobetasol spray as per Example 1 of the present invention and (b) Clobex® Spray

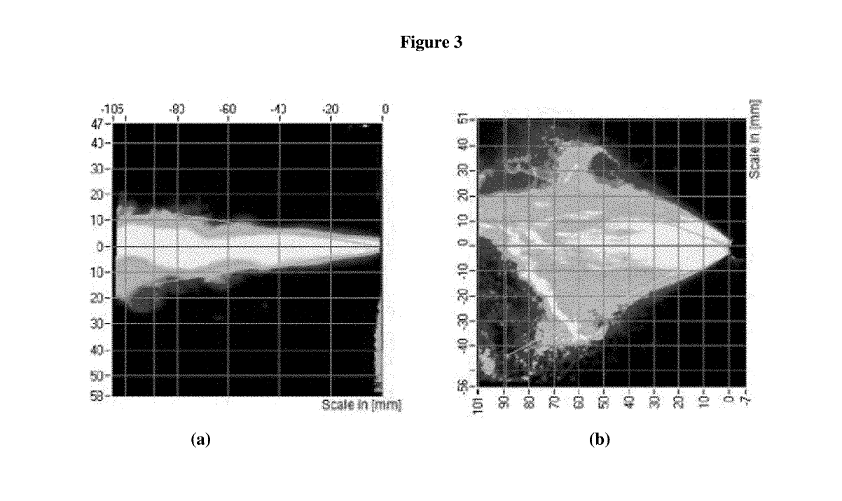

### DETAILED DESCRIPTION OF THE INVENTION

A first aspect of the present invention provides a halobetasol topical spray system comprising:
(i) a composition comprising halobetasol;
(ii) a dispensing system comprising:
   (a) a pressurized container comprising the composition and
   (b) a metered valve assembly comprising a metered valve having a stem connected to an actuator,
      wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume angle ranging from about 5° to about 40°.

In an embodiment of the above aspect, the plume angle is in a range of about 10° to about 30°. In a more particular embodiment of the above aspect, the plume angle is in a range of about 10° to about 25°.

In another embodiment of the above aspect, the plume angle is measured by SprayVIEW® instrument.

In another embodiment of the above aspect, the droplets are dispensed from a distance of about 3.5 inches.

A second aspect of the present invention provides a halobetasol topical spray system comprising:
(i) a composition comprising halobetasol;
(ii) a dispensing system comprising:
   (a) a pressurized container comprising the composition and
   (b) a metered valve assembly comprising a metered valve having a stem connected to an actuator,
      wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume width ranging from about 10 mm to about 65 mm.

In an embodiment of the above aspect, the plume width is in a range of about 15 mm to about 50 mm. In a more particular embodiment of the above aspect, the plume width is in a range of about 20 mm to about 40 mm.

In another embodiment of the above aspect, the plume width is measured by SprayVIEW® instrument.

In another embodiment of the above aspect, the droplets are dispensed from a distance of about 3.5 inches.

A third aspect of the present invention provides a halobetasol topical spray system comprising:
(i) a composition comprising halobetasol;
(ii) a dispensing system comprising:
   (a) a pressurized container comprising the composition and
   (b) a metered valve assembly comprising a metered valve having a stem connected to an actuator,
      wherein the actuator has an insert having an orifice diameter in a range of about 0.2 mm to about 1.5 mm, wherein when the actuator is depressed for activation to expel the composition from the pressurized container in the form of droplets, wherein the droplets form a spray pattern characterized by having one or more features of: a major axis in a range of about 4 mm to about 100 mm, a minor axis in a range of about 4 mm to about 100 mm, and an ellipticity in a range of about 1.00 to about 3.00.

In an embodiment of the above aspect, the droplets form a spray pattern characterized by having one or more features of: a major axis in a range of about 10 mm to about 60 mm, a minor axis in a range of about 10 mm to 60 mm, and an ellipticity in a range of about 1.00 to about 2.00.

In a more particular embodiment of the above aspect, the droplets form a spray pattern characterized by having one or more features of: a major axis in a range of about 20 mm to about 35 mm, a minor axis in a range of about 20 mm to about 35 mm, and an ellipticity in a range of about 1.00 to about 1.60.

In another embodiment of the above aspect, the spray pattern is measured by SprayVIEW® instrument.

In an embodiment of the above aspect, the droplets are dispensed from a distance of about 3.5 inches.

A fourth aspect of the present invention provides a halobetasol topical spray system comprising:
(i) a composition comprising halobetasol;
(ii) a dispensing system comprising:
   (a) a pressurized container comprising the composition and
   (b) a metered valve assembly comprising a metered valve having a stem connected to an actuator,
      wherein the actuator has an insert having an orifice diameter in a range of about 0.2 mm to about 1.5 mm, wherein when the actuator is depressed for activation to expel the composition from the pressurized container in the form of droplets, wherein the droplets form a spray pattern characterized by having one or more features of: a Dₘₐₓ in a range of about 4 to about 100 mm, a Dₘᵢₙ in a range of about 4 mm to about 100 mm, and an ovality of about 1.00 to about 3.00.

In an embodiment of the above aspect, the droplets form a spray pattern characterized by having one or more features of: a Dₘₐₓ in a range of about 10 mm to about 60 mm, a Dₘᵢₙ in a range of about 10 mm to 60 mm, and an ovality in a range of about 1.00 to about 2.00.

In a more particular embodiment of the above aspect, the droplets form a spray pattern characterized by having one or more features of: a Dₘₐₓ in a range of about 20 mm to about 35 mm, a Dₘᵢₙ in a range of about 20 mm to 35 mm, and an ovality in a range of about 1.00 to about 1.60.

In another embodiment of the above aspect, the spray pattern is measured by SprayVIEW® instrument.

In an embodiment of the above aspect, the droplets are dispensed from a distance of about 3.5 inches.

A fifth aspect of the present invention provides a halobetasol topical spray system comprising:
(i) a composition comprising halobetasol;
(ii) a dispensing system comprising:
   (a) a pressurized container comprising the composition and
   (b) a metered valve assembly comprising a metered valve having a stem connected to an actuator,
      wherein the actuator has an insert having an orifice diameter in a range of about 0.2 mm to about 1.5 mm, wherein when the actuator is depressed for activation to expel the composition from the pressurized container in the form of droplets, wherein the droplets form a spray pattern characterized by an area from about 300 mm² to about 900 mm².

In an embodiment of the above aspect, the droplets form a spray pattern characterized by an area from about 400 mm² to about 900 mm².

In another embodiment of the above aspect, the spray pattern is measured by SprayVIEW® instrument.

In an embodiment of the above aspect, the droplets are dispensed from a distance of about 3.5 inches.

According to another embodiment of the above aspects, wherein the composition comprises from about 0.01% w/w to about 0.5% w/w of halobetasol based on the total weight of the composition. In a preferred embodiment, the composition comprises about 0.05% w/w of halobetasol based on the total weight of the composition.

According to another embodiment of the above aspects, the composition comprises halobetasol propionate.

According to another embodiment of the above aspects, the composition is a solution.

According to another embodiment of the above aspects, there is provided a method of treating a skin disorder by topically applying the topical spray system to the affected skin of a patient wherein the skin disorder is selected from the group consisting of corticosteroid responsive dermatoses including psoriasis, atopic dermatitis, sebopsoriasis, palmoplantaris, pustulosis, ichtyosis, dermatoses, eczema, rosacea, acne vulgaris, pruritis, seborrhea, skin cancers, inflammation, and combinations thereof.

In a particular embodiment of the above aspect, the skin disorder is corticosteroid responsive dermatoses including psoriasis and atopic dermatitis.

According to another embodiment of the above aspect, the treatment of the skin disorders comprises co-administration of at least one additional drug used to treat topical skin conditions.

According to another embodiment of the above aspect, the additional drugs are selected from the group comprising Vitamin D analogues such as calcipotriene and calcitriol, retinoids such as tazarotene, calcineurin inhibitors such as tacrolimus and pimecrolimus, salicylic acid, coal tar, anthralin, antimicrobials, or combinations thereof. The co-administration includes simultaneous or sequential administration of additional drugs. Alternatively, the additional drugs can be administered together in a single composition.

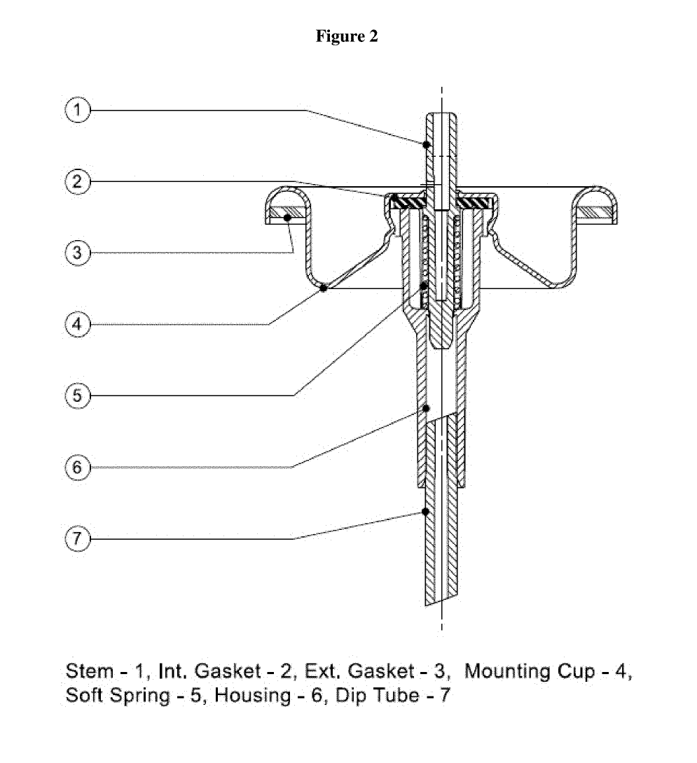

According to another embodiment of the above aspect, the metered valve assembly is further composed of a mounting cup attached to the pressurized container by crimping, a housing, a spring, a dip tube, and a gasket. The metered valve assembly is described in FIG. 2. Alternatively, the metered valve assembly may comprise a metering chamber.

According to another embodiment of the above aspect, the pressurized container may have a capacity ranging from about 5 g to about 200 g. In particular, the pressurized container may have a capacity of 15 g, 50 g, or 100 g.

According to another embodiment of the above aspect, the metered valve may have a capacity ranging from about 50 μL to about 200 μL. In particular, the metered valve may have a capacity ranging from about 120 μL to about 180 μL, more particularly about 150 μL.

According to another embodiment of the above aspect, the actuator is having a diameter ranging from about 5.0 mm to about 20.0 mm. In particular, the actuator diameter is in a range of about 10.0 mm to about 16.0 mm. More particularly, the actuator diameter is in a range of about 13.0 mm to about 15.0 mm.

According to another embodiment of the above aspect, the actuator has a height ranging from about 5.0 mm to about 20.0 mm. In particular, the actuator height is in a range of about 10.0 mm to about 16.0 mm. In particular, the actuator height is in a range of about 13.0 mm to about 15.0 mm.

The actuator has an insert having an orifice diameter in a range of about 0.2 mm to about 1.5 mm. In particular, the insert is having an orifice diameter in a range of about 0.2 mm to about 0.7 mm. More particularly, the insert is having an orifice diameter of about 0.5 mm.

According to another embodiment of the above aspect, the dip tube length ranges from about 50 mm to about 150 mm. For the pressurized container with a capacity of 15 g, the dip tube length ranges from about 50 mm to about 85 mm, for the pressurized container with a capacity of 50 g, the dip tube length ranges from about 90 mm to about 120 mm, and for the pressurized container with a capacity of 100 g, the dip tube length ranges from about 110 mm to about 150 mm.

The dimensions of the actuator, insert used in the present invention are designed in a way to deliver the composition with focused plume. The focused plume avoids the escape of droplets which may cause inhalation into the lungs leading to systemic side effects.

The components of the dispensing system used in the present invention are made of materials which are compatible with the composition contained inside and prevents degradation of the composition.

According to another embodiment of the above aspect, the pressurized container body is composed of a material selected from the group consisting of a metal such as aluminium or its alloy, stainless steel, or, tinplate; glass; polyester; ceramic; polyethylene terephthalate; or the like. In particular, the container body is composed of aluminium. Further, the container body is lined with an inert material to avoid any degradation of the composition due to interaction between the container body and the composition. The inert materials are selected from group consisting of resins such as acrylic, phenolic, epoxyphenolic, polyester, epoxy, or vinyl resins, fluoropolymers, such as perfluoroethylenepropylene copolymer fluororubber (FPM), perfluoroalkoxyalkanes, perfluoroalkoxyalkylenes, ethylene tetrafluoroethylene copolymer (EFTE), polytetrafluoroethylene (PTFE); ethylene-propylene diene monomer rubber (EPDM), polyamides, polyimides, polypropylene, polyethylene, or the like, in particular epoxyphenolic. The dip tube, the actuator, and the housing is composed of polyethylene or polypropylene or their copolymers. The stem is made up of a resin such as polyoxymethylene. The mounting cup made up of metals such as aluminum alloy. The dispensing system may further comprise gaskets or seals of materials composed of compatible materials fluoroelastomers, rubber such as nitrile rubber, fluororubber, ethylene-propylene diene monomer rubber, fluorinated ethylene-propylene copolymer.

The composition of the present invention has an internal pressure ranging from about 40 psig to about 150 psig, in particular from about 60 psig to about 100 psig.

According to another embodiment of the above aspect, the composition is stable.

According to another embodiment of the above aspect, the composition is non-foaming.

According to another embodiment of the above aspect, the composition is preservative-free.

According to another embodiment of the above aspect, the composition is stabilizer-free.

According to another embodiment of the above aspect, the composition is substantially anhydrous.

According to another embodiment of the above aspect, the composition further comprises a non-aqueous solvent, and a propellant, and optionally an emollient.

According to another embodiment of the above aspect, the composition consists essentially of halobetasol, a non-aqueous solvent, a propellant, and an emollient.

According to another embodiment of the above aspect, the composition consists of halobetasol, a non-aqueous solvent, a propellant, and an emollient.

According to another embodiment of the above aspect, the composition has a non-aqueous solvent to propellant ratio ranging from about 1:0.1 to about 1:15. In particular, the ratio ranges from about 1:0.2 to about 1:10, more particularly, the ratio ranges from about 1:0.4 to about 1:3.

According to another embodiment of above aspect, the composition further comprises an emollient.

According to another embodiment of above aspect, the composition has an emollient to non-aqueous solvent ratio ranging from about 1:1 to about 1:20, in particular from about 1:1 to about 1:10.

According to another embodiment of above aspect, the composition has an emollient to propellant ratio ranging from about 1:2 to about 1:10.

According to another embodiment of the above aspect, the composition is applied onto the affected skin twice daily for two consecutive weeks for treating skin disorders. In particular, the skin disorder is corticosteroid responsive dermatoses, more particularly corticosteroid responsive dermatoses is psoriasis and atopic dermatitis. For moderate to severe plaque psoriasis, treatment may be extended for an additional two weeks for localized lesions that have not sufficiently improved after the initial two weeks treatment. Furthermore, the topical spray composition is applied onto the affected skin twice daily for not more than four weeks for treating skin disorders.

According to another embodiment of the above aspect, the composition is a touch-free topical spray composition and requires no rubbing-in when said composition is applied to affected skin.

According to another embodiment of the above aspect, the composition is applied onto the affected skin for not more than 75 sprays per application for treating corticosteroid responsive dermatoses. Particularly, the composition is applied onto the affected skin for not more than about 35 sprays per application for treating corticosteroid responsive dermatoses when the average shot weight ranges from about 90 mg to about 110 mg. Alternatively, the composition is applied onto the affected skin for not more than about 44 sprays per application for treating corticosteroid responsive dermatoses when the average shot weight ranges from about 90 mg to about 110 mg.

According to another embodiment of the above aspect, the composition is applied onto the affected skin for not more than 150 sprays per day for treating corticosteroid responsive dermatoses. Particularly, the composition is applied onto the affected skin for not more than about 70 sprays per day for treating corticosteroid responsive dermatoses when the average shot weight ranges from about 90 mg to about 110 mg. Alternatively, the composition is applied onto the affected skin for not more than about 88 sprays per day for treating corticosteroid responsive dermatoses when the average shot weight ranges from about 65 mg to about 95 mg.

According to another embodiment of the above aspect, the composition of halobetasol is sprayed onto the affected skin for not more than 50 g per week for treating corticosteroid responsive dermatoses.

According to another embodiment of the above aspect, the dispensing system upon each actuation delivers an average shot weight of topical spray composition of halobetasol in a range of from about 50 mg to about 150 mg. In a preferred embodiment, the average shot weight ranges from about 75 mg to about 125 mg. In a more preferred embodiment, the average shot weight ranges from about 90 mg to about 110 mg. Alternatively, the average shot weight may also range from about 65 mg to about 95 mg.

According to another embodiment of the above aspect, the composition is delivered through a dispensing system in the form of droplets.

According to another embodiment of the above aspect, the droplets are having a D₉₀ of less than 150 μm. In a particular embodiment, the droplets have a D₉₀ of less than 100 μm. In a more particular embodiment, the droplets have a D₉₀ of less than 50 μm. In an even more particular embodiment, the droplets have a D₉₀ from about 20 μm to about 50 μm.

According to another embodiment of the above aspect, the droplets are having a D₅₀ of less than 80 μm. In a particular embodiment, the droplets have a D₅₀ of less than 50 μm. In a more particular embodiment, the droplets have a D₅₀ of less than 30 μm. In an even more particular embodiment, the droplets have a D₅₀ from about 5 μm to about 50 μm.

According to another embodiment of the above aspect, the droplets are having a D₁₀ of less than 40 μm. In a particular embodiment, the droplets have a D₁₀ of less than 30 μm. In a more particular embodiment, the droplets have a D₁₀ of less than 20 μm. In an even more particular embodiment, the droplets have a D₁₀ from about 7 μm to about 12 μm.

According to another embodiment of the above aspect, the droplets are having a droplet size distribution of D₉₀ of less than 150 μm, a D₅₀ of less than 80 μm, a D₁₀ of less than 40 μm.

According to another embodiment of the above aspect, the composition is delivered through a dispensing system in the form of droplets, wherein more than 10% of the droplets are smaller than 10 μm in size.

According to another embodiment of the above aspect, the droplets are having a droplet span in a range of about 1.10 to about 1.40.

According to another embodiment of the above aspect, the dispensing system delivers a metered dose of halobetasol upon each actuation.

A sixth aspect of the present invention provides a process for the preparation of a composition of halobetasol, wherein the process comprises the steps of:
(a) dissolving halobetasol in one portion of a non-aqueous solvent;
(b) optionally mixing an emollient and another portion of the non-aqueous solvent into the solution of step (a) to form a solution;
(c) dispensing the solution of step (b) in a pressurized container;
(d) crimping the container of step (c) with a metered valve assembly;
(e) charging the propellant in the container of step (d); and
(f) fitting an actuator on the container of step (e).

One of the embodiment of the present invention provides a topical spray system of halobetasol for topical administration to a subject having a corticosteroid responsive dermatosis comprising i) a composition comprising about 0.05% w/w of halobetasol, based on the total weight of the composition and (ii) a dispensing system, wherein the dispensing system delivers the composition to the skin of the subject, and wherein the topical spray system is effective in penetration of the halobetasol into the epidermis or dermis of the skin of the subject where the halobetasol has its effect on the corticosteroid responsive dermatosis.

Another embodiment of the present invention provides a topical spray system of halobetasol for topical administration to a subject having a corticosteroid responsive dermatosis comprising i) a composition comprising about 0.05% w/w of halobetasol, based on the total weight of the composition and (ii) a dispensing system, wherein the dispensing system delivers the composition to the skin of the subject, such that topical spray system exhibits a high level of penetration into the epidermis and/or the dermis of the skin of the subject.

Another embodiment of the present invention provides a topical spray system of halobetasol for topical application to a subject having a corticosteroid responsive dermatosis comprising i) a composition comprising about 0.05% w/w of halobetasol, based on the total weight of the composition (ii) a dispensing system, wherein the dispensing system delivers the composition to the skin of the subject, and wherein 24 hours after administration to the subject with the topical spray system, more than 10% w/w of halobetasol has penetrated into the epidermis and the dermis based on the total weight of the applied dose. Preferably, more than 15% w/w of halobetasol has penetrated into the epidermis and the dermis based on the total weight of the applied dose. More preferably, more than 20% w/w of halobetasol has penetrated into the epidermis and the dermis based on the total weight of the applied dose. Even more preferably, more than 25% w/w of halobetasol has penetrated into the epidermis and the dermis based on the total weight of the applied dose. Even much more preferably, more than 30% w/w of halobetasol has penetrated into the epidermis and the dermis based on the total weight of the applied dose.

Another embodiment of the present invention provides a method of treating corticosteroid responsive dermatoses in a subject in need thereof comprising topically spraying a composition onto the subject's skin using a dispensing system comprising the composition, wherein the method provides an effective amount of halobetasol at a target site for the corticosteroid responsive dermatoses, wherein the target site is selected from an epidermis layer, a dermis layer, or combination thereof. Preferably, the effective amount of halobetasol in epidermis and dermis is at a concentration of at least 10 times higher than the marketed halobetasol cream formulation.

Another embodiment of the present invention provides a method of penetrating halobetasol into the dermis and/or epidermis of a patient in need thereof, comprising: spraying a composition of halobetasol from a dispensing system onto the skin of the patient, wherein the spraying provides droplets of the composition having a D₉₀ of less than 150 μm.

Another embodiment of the present invention provides a method of penetrating halobetasol into skin layers of patient, comprising topically administering to the patient a composition comprising halobetasol using a dispensing system, and wherein the composition comprises about 0.05% w/w of halobetasol based on the total weight of the composition.

Another embodiment of the present invention provides a method of treating skin disorders in a patient in need thereof comprising topically spraying a composition using a dispensing system, wherein upon topically spraying the ratio of amount of halobetasol retained into the skin layers to the amount of halobetasol remained unabsorbed onto the skin is at least 0.5. The skin layers include stratus corneum, epidermis and dermis.

Another embodiment of the present invention provides a method of treating skin disorders in a patient in need thereof comprising topically spraying a composition using a dispensing system, wherein upon topically spraying the amount of halobetasol retained into the skin layers is at least 10 times higher than the marketed halobetasol cream formulation. The skin layers include stratus corneum, epidermis and dermis.

A seventh aspect of the present invention provides a dispensing system comprising:
(a) a pressurized container comprising a pharmaceutical composition and
(b) a metered valve assembly comprising a metered valve having a stem connected to an actuator,
   wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume angle ranging from about 5° to about 40°.

An eighth aspect of the present invention provides a dispensing system comprising:
(a) a pressurized container comprising a pharmaceutical composition and
(b) a metered valve assembly comprising a metered valve having a stem connected to an actuator,
   wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume width ranging from about 10 mm to about 65 mm.

A ninth aspect of the present invention provides a dispensing system comprising:
(a) a pressurized container comprising a pharmaceutical composition and
(b) a metered valve assembly comprising a metered valve having a stem connected to an actuator,
   wherein the actuator has an insert having an orifice diameter in a range of about 0.2 mm to about 1.5 mm, wherein when the actuator is depressed for activation to expel the composition from the pressurized container in the form of droplets, wherein the droplets form a spray pattern characterized by having one or more features of: a major axis in a range of about 4 mm to about 100 mm, a minor axis in a range of about 4 mm to about 100 mm, and an ellipticity in a range of about 1.00 to about 3.00.

A tenth aspect of the present invention provides a dispensing system comprising:
(a) a pressurized container comprising a pharmaceutical composition and
(b) a metered valve assembly comprising a metered valve having a stem connected to an actuator,
   wherein the actuator has an insert having an orifice diameter in a range of about 0.2 mm to about 1.5 mm, wherein when the actuator is depressed for activation to expel the composition from the pressurized container in the form of droplets, wherein the droplets form a spray pattern characterized by having one or more features of: a Dₘₐₓ in a range of about 4 to about 100 mm, a Dₘᵢₙ in a range of about 4 mm to about 100 mm, and an ovality of about 1.00 to about 3.00.

According to an embodiment of the above aspects, the composition comprises one or more propellants.

According to another embodiment of the above aspects, the composition further comprises one or more emollients and a non-aqueous solvent.

According to another embodiment of the above aspects, the composition comprises halobetasol propionate.

The term “topical,” as used herein, refers to a composition meant for application to the skin, scalp, nail, or mucosal tissue.

The term “spray,” as used herein, means to dispense the composition as a mass or jet of droplets from a dispensing system.

The term “stable,” as used herein, means chemical stability of halobetasol in the dispensing system in both upright and inverted positions, wherein not more than 5% w/w of total related substances are formed on storage at 40° C. and 75% relative humidity or at 25° C. and 60% relative humidity for a period of at least one month to the extent necessary for sale and use of composition. More particularly, the compositions remain stable for at least three months. Even more particularly, the compositions remain stable for at least six months.

The term “halobetasol,” as used herein, includes halobetasol and its salts, esters, polymorphs, hydrates, solvates, prodrugs, chelates, and complexes. The preferred ester of halobetasol is halobetasol propionate. The composition of the present invention comprises halobetasol in an amount from about 0.01% w/w to about 0.5% w/w based on the total weight of the composition. Preferably, the composition of the present invention comprises halobetasol in an amount of about 0.05% w/w based on the total weight of the composition.

The composition is characterized by having a specific gravity of about 0.5 g/mL to about 0.8 g/mL. In a particular embodiment, the composition is characterized by having a specific gravity of about 0.6 g/mL to about 0.8 g/mL.

The specific gravity of the bulk solution of the composition ranges from about 0.6 g/mL to about 1.0 g/mL. In particular, it ranges from about 0.7 g/mL to about 0.9 g/mL.

The composition of the present invention has an internal pressure ranging from about 40 psig to about 150 psig, in particular from about 60 psig to about 100 psig.

The term “bulk solution,” as used herein, means halobetasol composition without a propellant. The halobetasol composition herein means a composition comprising halobetasol, a non-aqueous solvent, and optionally an emollient.

In the present invention, the ratio of the bulk solution to the propellant ranges from about 1:20 to about 20:1, particularly from about 1:10 to about 10:1, more particularly about 1:1.

The term “substantially anhydrous,” as used herein means that the content of free water in the composition is not more than 40% w/w, particularly not more than 30% w/w, more particularly not more than 15% w/w, based on the total weight of the composition.

The composition of the present invention is microbiologically stable.

The term “microbiologically stable,” as used herein means not more than 200 of the total aerobic microbial count (cfu/g), not more than 20 of the total combined yeast/moulds count (cfu/g), and absence of Staphylococcus aureus (per g) and Pseudomonas aeruginosa (per g), for at least one month to the extent necessary for sale and use of composition on storage at 40° C. and 75% relative humidity or at 25° C. and 60% relative humidity. More particularly, the compositions remain microbiologically stable for at least three months. Even more particularly, the compositions remain microbiologically stable for at least six months.

The term “D₅₀,” as used herein refers to the diameter of droplet for which 50% of the total liquid volume of sample consists of droplets of a smaller diameter (μm), also known as the mass median diameter. The present invention includes droplets having D₅₀ of less than about 80 μm. Particularly, the droplets have a D₅₀ of less than about 50 μm. More particularly, the droplets have a D₅₀ of less than about 30 μm.

The term “D₉₀,” as used herein refers the diameter of droplet for which 90% of the total liquid volume of sample consists of droplets of a smaller diameter (m). The present invention includes droplets having D₉₀ of less than about 150 μm. Particularly, the droplets have a D₉₀ of less than about 100 μm. More particularly, the droplets have a D₉₀ of less than about 50 μm.

The term “D₁₀,” as used herein refers to the diameter of droplet for which 10% of the total liquid volume of sample consists of droplets of a smaller diameter (μm). The present invention includes droplets having D₁₀ the droplets are having a D₁₀ of less than 40 μm. Particularly, the droplets have a D₁₀ of less than 30 μm. More particularly, the droplets have a D₁₀ of less than 20 μm.

The term “span,” as used herein, refers to the measurement of the width of the distribution. The smaller the value, the narrower the distribution. Span is calculated as (D₉₀−D₁₀)/D₅₀.

The D₅₀, D₉₀, D₁₀, are measured by using a Spraytec® instrument in which the distance between the actuator of the dispensing system and laser was kept about 4 inch.

The term “about” as used herein, refers to any value which lies within the range defined by a variation of up to ±10% of the value.

The term “ellipticity,” as used herein, refers to the ratio of major axis to minor axis of the spray pattern.

The term “major axis,” as used herein, refers to the largest chord, in mm, that can be drawn within the spray pattern.

The term “minor axis,” as used herein, refers to the minimum chord, in mm, that can be drawn within the spray pattern.

The term “ovality,” as used herein, refers to the ratio of Dₘₐₓ and Dₘᵢₙ. The ovality of the spray pattern indicates whether the spray is symmetrical.

The term “Dₘₐₓ,” as used herein, refers to the largest chord, in mm, that can be drawn within the spray pattern that crosses the COMw (i.e., center of mass of the spray pattern) in base units.

The term “Dₘᵢₙ,” as used herein, refers to the smallest chord, in mm, that can be drawn within the spray pattern that crosses the COMw in base units.

The term “COMw,” as used herein, refers to the center of mass of the detected spray pattern, where each pixel's intensity is taken into account.

Spray pattern is characterized by major axis, minor axis, ellipticity, Dₘₐₓ, Dₘᵢₙ and ovality. Spray pattern is measured by using SprayVIEW® instrument. In the present invention, the major axis ranges from about 5 mm to about 100 mm, minor axis ranges from about 4 mm to about 100 mm, and ellipticity ranges from about 1.00 to about 3.00. Further, specifically Dₘₐₓ ranges from about 5 mm to about 100 mm, a Dₘᵢₙ ranges from about 4 mm to about 100 mm, and ovality ranges from about 1.00 to about 3.00.

The term “plume angle,” as used herein, refers to the angle of the emitted plume measured from the vertex of the spray cone and spray nozzle. It is measured by using SprayVIEW® instrument and it ranges from about 5° to about 40°. In particular, it ranges from about from about 10° to about 30°, more particularly, it ranges from about from about 15° to about 25° when measured at a distance of about 3.5 inches.

The term “plume width,” as used herein, refers to the width of the plume at a given distance from the spray nozzle. It is measured by using SprayVIEW® instrument and it ranges from about 10 mm to about 65 mm, in particular from about 15 mm to about 50 mm, more particularly from about 20 mm to about 40 mm when measured at a distance of about 3.5 inches.

The term “emollient,” as used herein refers to a substance that helps to retain the skin moisture and also helps to control the rate of evaporation and the tackiness of the composition. It also helps to soften the thickened epidermis of the psoriatic plaques. Additionally, emollients provide a softening or soothing effect on the skin surface. Suitable examples of emollients are selected from the group consisting of fatty acid triglycerides such as mixture of caprylic and capric triglycerides (Crodamol™ GTCC-LQ, Miglyol™, Captex™ Labrafac™, Lipophile™ WL), palmitic triglyceride, oleic triglyceride, caprylic triglyceride, capric triglyceride, and linoleic triglyceride; fatty acid esters such as isopropyl myristate, isopropyl palmitate, dibutyl adipate, and dibutyl phthalate; polyhydric alcohols such as propylene glycol, butylene glycol, polyethylene glycol, glycerol, and sorbitol; fatty acids such as oleic acid and stearic acid; oils such as mineral oil, lanolin oil, coconut oil, cocoa butter, olive oil, jojoba oil, and castor oil; cyclomethicone; hydrogenated lanolin; waxes; lecithin; or mixtures thereof. Preferably, the emollient of the present invention is selected from the group consisting of fatty acid triglycerides, fatty acid esters, and polyhydric alcohols. More preferably, the emollient of the present invention is isopropyl palmitate. The emollient used in the present invention ranges from about 1% w/w to about 45% w/w based on the total weight of the composition. Particularly, the emollient used in the present invention ranges from about 1% w/w to about 30% w/w based on the total weight of the composition. More particularly, the emollient used in the present invention ranges from about 1% w/w to about 20% w/w based on the total weight of the composition. Even more particularly, the emollient used in the present invention ranges from about 1% w/w to about 10% w/w based on the total weight of the composition. Even more particularly, the emollient used in the present invention ranges from about 6% w/w to about 8% w/w based on the total weight of the composition.

The term “non-aqueous solvent,” as used herein refers to the solvent used to dissolve halobetasol. Suitable non-aqueous solvents are selected from the group consisting of ethyl alcohol, isopropyl alcohol, propylene glycol, butanediol, pentanediol, hexanediol, triethylene glycol, tetraethylene glycol, dipropylene glycol, dibutylene glycol, glycerin, dimethyl isosorbide, tetrahydro furfuryl alcohol polyethylene glycol ether, N-methyl-2-pyrrolidone, 1-methyl-2-pyrrolidinone, dimethylsulfoxide, dimethylacetamide, lactic acid, glycolic acid, methylene chloride, methyl-ethyl-ketone, ethyl acetate, methylene dimethyl ether, or mixtures thereof. In particular, ethyl alcohol is dehydrated ethyl alcohol. The non-aqueous solvent used in the present invention ranges from about 1% w/w to about 95% w/w based on the total weight of the composition. Particularly, the non-aqueous solvent is present from about 10% w/w to about 70% w/w based on the total weight of the composition. More particularly, the non-aqueous solvent is present from about 20% w/w to about 50% w/w based on the total weight of the composition. Even, more particularly, non-aqueous solvent is present from about 38% w/w to about 43% w/w based on the total weight of the composition.

The term “propellant,” as used herein refers to the substance that helps in propelling the composition out of the container. Suitable examples of propellants are selected from the group consisting of conventional, non-ozone depleting hydrocarbon propellants. These include propane butane, isobutane, cyclopropane, liquefied petroleum gas, 1,1,1,2-tetrafluoroethane, 1,1,1,2,3,3,3-heptafluoropropane, 1,1-difluoroethane, 1,1,1,3,3,3-hexafluoropropane, or mixtures thereof. Fluorocarbon gas and may also be used as propellants. The propellant is used in the present invention ranges from about 5% w/w to about 95% w/w based on the total weight of the composition. Particularly, the propellant is used in the present invention ranges from about 20% w/w to about 80% w/w based on the total weight of the composition. More particularly, the propellant is used in the present invention ranges from about 30% w/w to about 70% w/w based on the total weight of the composition.

The topical spray composition of the present invention further comprises solubilizers, permeation enhancers, film-formers, plasticizers, antioxidants, pH-adjusting agents, or mixtures thereof.

The solubilizer is a substance that aids in the dissolution or dispersion of halobetasol in the composition. Suitable solubilizers are selected from the group consisting of polyhydric alcohols such as propylene glycol and polyethylene glycol; fatty acids such as oleic acid and stearic acid; non-ionic and ionic surfactants such as polyoxyethyl-sorbitan-fatty acid esters such as polysorbates, ethers of sugars, ethoxylated fatty alcohols, sodium lauryl sulfate, taurocholic acid, lecithin and Labrasol™; vitamin E; vitamin E TPGS (tocopheryl polyethylene glycol 1000 succinate); caprylic triglyceride; capric triglyceride; or combinations thereof.

The permeation enhancer is a substance used to enhance the penetration rate of halobetasol through the skin. Suitable permeation enhancers are selected from the group consisting of lipophilic solvents such as dimethyl sulfoxide and dimethyl formamide; non-ionic and ionic surfactants such as polyoxyethyl-sorbitan-fatty acid esters such as polysorbates, ethers of sugars, ethoxylated fatty alcohols, sodium lauryl sulfate, taurocholic acid, lecithin and labrasol; fatty acid esters such as isopropyl myristate and isopropyl palmitate; fatty acids such as oleic acid and stearic acid; polyhydric alcohols such as propylene glycol and polyethylene glycol e.g., polyethylene glycol 400; Transcutol®; essential oils e.g., menthol; caprylic/capric triglycerides; or combinations thereof.

The film-former is a substance that forms a stable film on a topical surface when applied. Suitable film-formers are selected from the group consisting of acrylic polymers or copolymers such as methacrylic acid copolymers; cellulose derivatives such as cellulose acetate, hydroxypropyl methyl cellulose, hydroxy ethyl cellulose, methyl cellulose and ethyl cellulose; polyvinyl acetate; polyvinyl alcohol; povidone; povidone vinyl acetate; or combinations thereof. These film-formers can partially dissolve on exposure to moisture from the skin or air, the dissolution resulting in the formation of a porous film. This porosity can be enhanced by including additional water-soluble additives. The water-soluble additive is preferably propylene glycol, sodium lauryl sulphate, poloxamers, polyoxyl 35 castor oil, polyoxyl 40 hydrogenated castor oil, cetomacrogol, polyethylene glycol, transcutol, or combinations thereof.

The plasticizer is a substance that aids the composition in forming a flexible, adherent film on the skin. Suitable plasticizers are selected from the group consisting of citric acid esters, dimethyl isosorbide, castor oil, propylene glycol, polyethylene glycol, glycerol, oleic acid, citric acid, phosphate esters, fatty acid esters, glycol derivatives, hydrocarbons and their derivatives, adipic acid, butanediol polyesters, diethyl phthalate, dibutyl phthalate, chlorinated paraffins, or combinations thereof.

Suitable antioxidants are selected from the group consisting of butylated hydroxyl anisole, butylated hydroxy toluene, sodium metabisulfite, ascorbic acid, ascorbyl palmitate, thiourea, acetylcysteine, dithiothreitol, cysteine hydrochloride, propyl gallate, tocopherol, or combinations thereof.

Suitable pH-adjusting agents are selected from the group consisting of pharmaceutically acceptable organic or inorganic acids or bases such as sodium hydroxide, tromethamine, hydrochloric acid, citric acid, inorganic oxides, inorganic salts of weak acids, or combinations thereof.

In the present invention, the dispensing system comprises a pressurized container and a metered valve assembly as described in FIG. 2. The dispensing system may further comprise a dust cap or protective cap which is fitted onto the container to shield the contents of the container from the outside environment and undesirable pressing of actuator. The metered valve assembly comprises a mounting cup, a housing, a spring, a dip tube, a metered valve having stem connected to an actuator. The mounting cup can have an internal (Int.) gasket and an external (Ext.) gasket. Alternatively, the metered valve may have a metering chamber.

As described in FIG. 2, the metered valve assembly has a housing (6) which combines and accommodate the valve components from dip tube (7) to stem (1). Before actuation of the valve stem (1), the valve is at rest. The filling paths in the inner section of the valve stem (1) bridge the flexible section of housing, allowing free transfer through the dip tube (7) of the contents of the housing (6) and the bulk contents of the container. On actuation, the actuator depresses the valve stem (1), brings the internal cylindrical portion of the stem (1) into a sealing contact with the housing (6).

External gasket (3) in mounting cup (4) is meant for perfect sealing valve with the container. At this stage the exit hole of the stem (1) is still covered by the internal gasket (2). The metered volume to be delivered is isolated from the halobetasol composition of the container. Continued depression of the stem (1) moves the exit hole in the stem in to the isolated volume of housing (6).

The composition in the housing (6) contains a significant proportion of a volatile propellant gas in thermal equilibrium. When the exit hole enters the housing (6), the contents of the housing (6) are connected to the atmosphere, which is at a substantially lower pressure. The liquefied propellant boils vigorously to restore equilibrium and in so doing evacuates the halobetasol composition from the housing (6).

When the depressing force on the stem is removed the spring (5) returns the stem (1) back towards the rest position. The exit orifice in the stem (1) is first covered by the wall of the internal gasket (2), sealing off the flow-path to atmosphere. Further travel allows the filling paths in the inner section of the stem (1), to bridge the flexible section of the housing (6).

The residual contents of the housing (6) are typically propellant vapour which are at low temperature and pressure. As the contents of the container are at higher pressure than the chamber, liquid is forced in to the housing (6), via the filling paths in the inner section of the stem.

The metered valve dispenses a metered quantity of composition with each actuation of the actuator. The metered quantity avoids under-dosing or overdosing that may lead to undesirable side effects.

The distance between the base of actuator to the place where it fits on to the wall of the stem is about 0.5 mm to about 10 mm, particularly 7.8 mm.

Specifically, in the present invention, the pressurized container has a capacity of 50 g and the metered valve has a capacity of 150 μL. Further, the actuator is provided with an insert having an orifice diameter of about 0.5 mm and the actuator has a diameter of about 13.9 mm with a height of about 14.0 mm.

The dimensions of the actuator, insert, pressure within the container, and specific gravity of the composition determine the spray pattern characteristics such that the spray on expulsion forms a focused coverage area and also ensures that the size of droplets are sufficiently small to form a uniform spray with a characteristic of ‘no-running’ and yet sufficiently large to ensure that the droplets do not form a fine mist that can be inhaled.

The metered valve dispenses a metered quantity of composition with each actuation of the actuator. The metered quantity avoids under-dosing or overdosing that may lead to undesirable side effects. The dispensing system upon each actuation delivers an average shot weight in a range of from about 50 mg to about 150 mg. In particular, the average shot weight ranges from about 75 mg to about 125 mg. More particularly, the average shot weight ranges from about 90 mg to about 110 mg. The dispensing system of the present invention ensures consistent shot weight upon each actuation. The dispensing system of the present invention further ensures that with each actuation, dose uniformity is maintained. The dispensing system of the present invention also complies with other USP regulatory requirements including the leakage test.

Containers can be made from materials selected from the group consisting of stainless steel, aluminum, plastic, and glass. The plastic container can be made up of high density polyethylene (HDPE). The containers can be coated with an inert inner lining of epoxy-phenolic resins, epoxy-urea-formaldehyde resins, polytetrafluroethylene (PTFE), perfluoroethylenepropylene (PFEP), perfluoroalkoxyalkane (PFA), ethylene tetrafluoroethylene (ETFE), polyvinylidene fluoride (PVDF), chlorinated ethylene tetrafluoroethylene, or another coating treatment that creates a barrier to chemical interaction between the composition and the container. The dust cap can be made of any suitable material such as plastic or metal.

The actuation force value ranges from about 10 N to about 40 N, in particular actuation force value ranges from about 20 N to about 30 N. The term “actuation force value,” refers to any the force that presses the actuator to deliver the composition through its orifice.

The amount of halobetasol may depend upon the purpose for which the composition is to be applied. For example, the dosage and frequency of application can vary depending upon the type and severity of the topical condition.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Example 1

| Ingredient | Quantity (% w/w) |
|---|---|
| Halobetasol propionate | 0.05 |
| Ethyl alcohol | 42.65 |
| Isopropyl palmitate | 7.30 |
| Liquefied petroleum gas | 50.00 |

Procedure:
1. Halobetasol propionate was dissolved into a portion of ethyl alcohol with stirring.
2. Isopropyl palmitate was added while stirring into the solution of step 1.
3. The remaining quantity of ethyl alcohol was added into the solution of step 2 and mixed.
4. The solution of step 3 was filled into an aluminum container with an inert liner.
5. The container of step 4 was then crimped with a metered valve as described in FIG. 2.
6. Liquefied petroleum gas was then charged into the container of step 5.
7. The container of step 6 was then fitted with an actuator which is then fitted with a dust cap to form halobetasol spray.

### Example 2

| Ingredients | Quantity (% w/w) |
|---|---|
| Halobetasol propionate | 0.05 |
| Ethyl alcohol | 52.65 |
| Isopropyl palimitate | 7.30 |
| Liquefied petroleum gas | 40.00 |

Procedure:
1. Halobetasol propionate is dissolved into a portion of ethyl alcohol with stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Liquefied petroleum gas is then charged into the container of step 5.
7. The container of step 6 is then fitted with an actuator which is then fitted with a dust cap to form halobetasol spray.

### Example 3

| Ingredients | Quantity (% w/w) |
|---|---|
| Halobetasol propionate | 0.05 |
| Ethyl alcohol | 42.65 |
| Isopropyl palmitate | 7.30 |
| Isobutane | 50.00 |

Procedure:
1. Halobetasol propionate is dissolved into a portion of ethyl alcohol with stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Isobutane is then charged into the container of step 5.
7. The container of step 6 is then fitted with an actuator which is then fitted with a dust cap to form halobetasol spray.

### Example 4

| Ingredients | Quantity (% w/w) |
|---|---|
| Halobetasol propionate | 0.05 |
| Ethyl alcohol | 32.65 |
| Isopropyl palmitate | 7.30 |
| Isobutane | 60.00 |

Procedure:
1. Halobetasol propionate is dissolved into a portion of ethyl alcohol with stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Isobutane is then charged into the container of step 5.
7. The container of step 6 is then fitted with an actuator which is then fitted with a dust cap to form halobetasol spray.

### Example 5

| Ingredients | Quantity (% w/w) |
|---|---|
| Halobetasol propionate | 0.05 |
| Ethyl alcohol | 32.65 |
| Isopropyl myristate | 7.30 |
| n-Butane | 60.00 |

Procedure:
1. Halobetasol propionate is dissolved into a portion of ethyl alcohol with stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. n-Butane is then charged into the container of step 5.
7. The container of step 6 is then fitted with an actuator which is then fitted with a dust cap to form halobetasol spray.

### Example 6

| Ingredients | Quantity (% w/w) |
|---|---|
| Halobetasol propionate | 0.05 |
| Ethyl alcohol | 52.65 |
| Isopropyl myristate | 7.30 |
| Liquefied petroleum gas | 40.00 |

Procedure:
1. Halobetasol propionate is dissolved into a portion of ethyl alcohol with stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner as described in FIG. 2.
5. The container of step 4 is then crimped with a metered valve.
6. Liquefied petroleum gas is then charged into the container of step 5.
7. The container of step 6 is then fitted with an actuator which is then fitted with a dust cap to form halobetasol spray.

### Example 7

| Ingredients | Quantity (% w/w) |
|---|---|
| Halobetasol propionate | 0.05 |
| Ethyl alcohol | 32.65 |
| Isopropyl palmitate | 7.30 |
| Isobutane | 60.00 |

Procedure:
1. Halobetasol propionate is dissolved into a portion of ethyl alcohol with stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner as described in FIG. 2.
5. The container of step 4 is then crimped with a metered valve.
6. Isobutane is then charged into the container of step 5.
7. The container of step 6 is then fitted with an actuator which is then fitted with a dust cap to form halobetasol spray.

### Example 8

| Ingredients | Quantity (% w/w) |
|---|---|
| Halobetasol propionate | 0.05 |
| Ethyl alcohol | 19.95 |
| Propylene glycol | 30.00 |
| Isobutane | 50.00 |

Procedure:
1. Halobetasol propionate is dissolved into a portion of ethyl alcohol with stirring.
2. Propylene glycol is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Isobutane is then charged into the container of step 5.
7. The container of step 6 is then fitted with an actuator which is then fitted with a dust cap to form halobetasol spray.

### Example 9

| Ingredients | Quantity (% w/w) |
|---|---|
| Halobetasol propionate | 0.05 |
| Ethyl alcohol | 19.95 |
| Propylene glycol | 30.00 |
| Liquefied petroleum gas | 50.00 |

Procedure:
1. Halobetasol propionate is dissolved into a portion of ethyl alcohol with stirring.
2. Propylene glycol is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Liquefied petroleum gas is then charged into the container of step 5.
7. The container of step 6 is then fitted with an actuator which is then fitted with a dust cap to form halobetasol spray.

### Example 10

| Ingredients | Quantity (% w/w) |
|---|---|
| Halobetasol propionate | 0.05 |
| Ethyl alcohol | 20.00 |
| Caprylic and capric triglycerides | 29.95 |
| (Crodamol ™ GTCC-LQ) | |
| Isobutane | 50.00 |

Procedure:
1. Halobetasol propionate is dissolved into a portion of ethyl alcohol with stirring.
2. Crodamol™ GTCC-LQ is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Isobutane is then charged into the container of step 5.
7. The container of step 6 is then fitted with an actuator which is then fitted with a dust cap to form halobetasol spray.

### Example 11

| Ingredients | Quantity (% w/w) |
|---|---|
| Halobetasol propionate | 0.05 |
| Ethyl alcohol | 20.00 |
| Caprylic and capric triglycerides | 29.95 |
| (Crodamol ™ GTCC-LQ) | |
| Liquefied petroleum gas | 50.00 |

Procedure:
1. Halobetasol propionate is dissolved into a portion of ethyl alcohol with stirring.
2. Crodamol™ GTCC-LQ is added while stirring into the solution of step 1.
3. The remaining quantity of ethyl alcohol is added into the solution of step 2 and mixed.
4. The solution of step 3 is filled into an aluminum container with an inert liner.
5. The container of step 4 is then crimped with a metered valve as described in FIG. 2.
6. Liquefied petroleum gas is then charged into the container of step 5.
7. The container of step 6 is then fitted with an actuator which is then fitted with a dust cap to form halobetasol spray.

### Droplet Size of Halobetasol Spray

The droplet size of Halobetasol spray of Example 1 (average shot weight=101.6 mg) and Clobex® spray (average shot weight=104 mg) was determined by Spraytec® instrument. Initial 6 sprays have been used for priming for Halobetasol spray of Example 1 and 4 sprays for Clobex® spray. Distance between the actuator and laser was kept at 4 inch. Data are collected for D10, D50, D90, and % <10 μm and provided in Table 1.

TABLE 1
Droplet size of Halobetasol spray of Example
1 and Clobex ® spray

| | Example 1 | Clobex ® Spray |
|---|---|---|
| Droplet Size | (Mean of 3 sprays) | (Mean of 4 sprays) |
| D(10) (μm) | 9.664 | 48.13 |
| D(50) (μm) | 18.60 | 96.17 |
| D(90) (μm) | 33.99 | 188.95 |
| Span | 1.308 | 1.47 |
| % <10 μm | 11.17 | 0.02 |

### Plume Geometry

The plume geometry of Halobetasol spray of Example 1 was determined for three sprays with an average shot weight of 103.4 mg in which initial 6 sprays were used for priming. Similarly, the plume geometry of Clobex® spray was determined for three sprays with an average shot weight of 104 mg in which initial five sprays were used for priming. The plume geometry of Halobetasol spray of Example 1 and Clobex® Spray was determined by SprayVIEW® with instrument parameters of velocity=70 mm/sec, acceleration=3000 mm/sec², frame=250, frame rate=500 frames/sec, stroke length=3.6 mm, and hold time=360 ms. Distance between the actuator orifice and laser was kept 3.5 inch. Plume geometry is characterised by the plume angle and plume width and is provided in Table 2.

TABLE 2
Plume geometry of Halobetasol spray of Example
1 and Clobex ® spray

| | Halobetasol spray | Clobex ® Spray |
|---|---|---|
| Parameter | (Mean of 3 sprays) | (Mean of 3 sprays) |
| Plume angle | 17.9° | 44.8° |
| Width (mm) | 27.97 | 73.47 |

The plume angle as well as plume width herein depicts the coverage area. A smaller plume angle and plume width observed with the halobetasol spray of the present invention illustrates the focused coverage area whereas a broader plume angle and plume width observed with the Clobex® spray illustrates the wider coverage area.

### Spray Pattern

The spray pattern of halobetasol spray of Example 1 was determined for three sprays with an average shot weight of 102.8 mg in which initial six sprays were used for priming. The Spray pattern was determined by SprayVIEW® with instrument parameters of velocity=70 mm/sec, acceleration=3000 mm/sec², frame=250, frame rate=500 frames/sec, stroke length=3.6 mm, and hold time=360 ms. Distance between the actuator orifice and laser was kept 3.5 inch.

Spray pattern is characterized by the Dₘₐₓ, Dₘᵢₙ, ovality, major axis, minor axis, and ellipticity and values are given in Table 3.

TABLE 3
Spray pattern of Halobetasol spray of Example 1

| | Value |
|---|---|
| Parameter | (Mean of 3 sprays) |
| Major axis (mm) | 27.84 |
| Minor axis (mm) | 25.93 |
| Ellipticity | 1.074 |
| Dₘᵢₙ (mm) | 25.68 |
| Dₘₐₓ (mm) | 28.73 |
| Ovality | 1.119 |
| Area (mm²) | 580.9 |

Spray pattern of Clobex® spray was not captured by the instrument, as a spray good enough to be captured by the equipment was not generated. A mist of fine droplets with non-uniform spray pattern was generated, which may be the reason for the same.

### Specific Gravity

Specific gravity was measured by using a relative density apparatus. The specific gravity of the bulk solution of Example 1 was found to be 0.794 g/mL at 25° C. The specific gravity of the final composition was found to be 0.634 g/mL considering the specific gravity of liquefied petroleum gas as 0.5277 g/mL at 25° C. The specific gravity of Clobex® spray composition was found to be 0.8425 g/mL at 25° C.

### Shot Weight Per Actuation (Valve Delivery)

The shot weight of the halobetasol spray as per Example 1 was determined for five dispensing systems with three sprays made for each dispensing system. The average of these 15 sprays was taken and found to be 103 mg per actuation. Further, when a target of 103 mg per actuation was set, the maximum % difference of individual dispensing system was determined to be 3.7 and the mean % difference of dispensing system was determined to be 2. The data ensures consistent shot weight upon each actuation with minimum variation.

### Delivered Dose Uniformity (DDU)

The Delivered Dose Uniformity (DDU) of the halobetasol spray prepared as per Example 1 was determined. Twenty sprays were made (10 from beginning and 10 from end of the doses) from each dispensing system and the amount of halobetasol propionate was determined in each spray. The same procedure was performed on 10 dispensing systems. The halobetasol content in the mean of beginning 10 sprays and end 10 sprays were determined to be 99.2% and 101.6% respectively. The mean halobetasol content from these 20 determinations was found to be 100.4%. The results ensure uniform delivery of the halobetasol upon each actuation with minimum variation.

### Leakage Test

12 aerosol containers were selected and the weight of each container to the nearest mg (W1) was noted. The date and time to the nearest half hour were recorded and the containers were charged into a humidity chamber maintained at 25±2° C./60°±5 R.H. by keeping each container in upright position for not less than 3 days (not less than 72 hours). The containers were removed from the humidity chamber and date and time to the nearest half hour at the time of removal from the humidity chamber were recorded. The weight of each container (W2) was noted to the nearest mg. The time T, in hours, was determined during which the containers were under test.

The leakage rate was calculated, in mg per year, of each container taken by the formula:

Leakage rate (mg/year)=365×24×(W1−W2)/T

### Where,

W1=weight of the container (in mg)
W2=weight of the container after leakage test (in mg)
T=Time point (in hours) for which the leakage test has been carried out.

All the containers were kept in a freezer for chilling to reduce the internal pressure for about 15 minutes. Each container was cut and all the contents of the container were removed. The container was thoroughly washed with methanol. All associated parts of container were retained as a unit and dry them in an air oven at 100° C. for about 5 minutes to ensure complete evaporation of methanol. The containers were cooled to room temperature and the weight of each empty container (as a unit) (W3) was recorded to the nearest mg. The net fill weight was determined as W1-W3 for each container.

The leakage rate (% year) was calculated using the following formula:

Leakage rate (% of net fill weight/year)=Leakage rate (mg/year)×100/W1−W3

Average leakage rate (% of net fill weight/year)=Sum of leak rate of each containers/Number of containers

where,
W1−W3=net fill weight (in mg)
W1=weight of the container (in mg)
W2=weight of the container after leakage test (in mg)
T=Time point (in hours) for which the leakage test has been carried out.

The Average % leakage rate was determined to be 0.9 and Maximum % Leakage/year of individual container was determined to be 1.1. The results complied with the USP limits.

### Stability Data

The halobetasol spray of Example 1 was stored at a temperature of 40° C. and a relative humidity (R.H.) of 75% for a period of three months and six months in both upright and inverted positions of the dispensing systems, and analyzed for halobetasol propionate content by an HPLC method. The results of the analysis are represented in Table 4.

TABLE 4
Stability Data for the Halobetasol Spray as per Example 1

| | | | Related substances | |
|---|---|---|---|---|
| | Assay (%) | | (% w/w) | |
| Time | Upright | Inverted | Upright | Inverted |
| Initial | 99.7 | | Not detected | |
| 3 months (40° C./75% R.H.) | 97.8 | 97.9 | 0.19 | 0.21 |
| 6 months (40° C./75% R.H.) | 99.0 | 97.8 | 0.35 | 0.34 |

From the above data, it is clear that the halobetasol composition remains stable for six months when stored at accelerated conditions of 40° C./75% R.H.

### Microbial Test

The halobetasol spray as per Example 1 was stored at a temperature of 40° C. and a relative humidity (R.H.) of 75% for a period of six months, and analyzed for microbial count (Table 5).

TABLE 5
Microbial Test

| | Total | Total | | |
|---|---|---|---|---|
| | aerobic | combined | Staphylococcus | Pseudomonos |
| | microbial | yeast/moulds | aureus | aeruginosa |
| | count (cfu/g) | count (cfu/g) | (per g) | (per g) |
| Initial | Less than 10 | Less than 10 | Absent | Absent |
| 6 months | Less than 10 | Less than 10 | Absent | Absent |
| (40° C./ | | | | |
| 75% R.H.) | | | | |

From the above data, it is clear that the halobetasol composition remains microbiologically stable for six months when stored at accelerated conditions of 40° C./75% R.H.

### Internal Pressure

The internal pressure of halobetasol composition of Example 1 was found to be 80 psig.

### In-Vitro Permeation Studies

The in-vitro skin permeation studies were performed on halobetasol spray of Example 1 (halobetasol propionate 0.05% w/w) and halobetasol cream formulation manufactured by Perrigo® (halobetasol propionate 0.05% w/w) using a Franz diffusion cell (FDC). For this, human cadaver skin from two different donors and 3 FDCs for each formulation were used. The following parameters were evaluated, amount of: analyte un-absorbed on skin through surface wash, analyte in stratum corneum through tape stripping, analyte absorbed in epidermis, analyte absorbed in dermis. Analysis was carried out with an HPLC method.

Before in vitro skin permeation test skin integrity was checked by electrical resistance method using precision component analyzer (LCR meter). Following parameters were set on Precision Component Analyzer, 1) Frequency: 1000 Hz (1 KHz), 2) AC databridge: 300 mV in parallel circuit mode.

Normal saline solution was filled in donor chamber. One electrode was inserted through sampling arm into centre of receptor chamber and another electrode was placed in saline solution over skin in donor chamber. The resistance values observed in KΩ or Ω were noted.

After skin integrity test, receptor solution was filled into receptor chamber. The assembly was kept on magnetic stirrer and heating circulator bath were switched ON. The skin was equilibrated until temperature on skin surface reaches at 32±1° C. Prior to dose application on skin surface, pre-dose sample (0.300 mL) was collected from each FDC and the receptor solution was replaced with stock receptor solution. For application of spray formulation, dispensing tube with actuator was placed on spray nozzle of can. Donor chamber of FDCs were occluded with parafilm before application of spray formulation. The dispensing tube was inserted into donor chamber through parafilm and single spray stroke were dispensed gently. The actual amount of halobetasol propionate in topical spray formulation was 50000 ng (in single spray). The parafilm were removed from the donor chamber after 10 minute of spray formulation application and were dipped into 5 mL of methanol. Similarly 100 μL of cream formulation was applied using positive displacement pipettes (here donor chamber were left unoccluded). The actual amount of halobetasol propionate in cream formulation was 47362 ng (in 100 μL of cream).

At each sampling time point, 0.300 mL of receptor solution were removed from centre of receptor chamber using a dosing syringe and was replaced with fresh stock of receptor solution up to the mark on the sampling arm. At the end of the 24 hour duration, the surface of the skin was washed with 1 mL, five times (5 mL) in succession, with methanol to collect un-absorbed formulation from the skin surface and was pooled. These surfaces washed samples were further diluted two times with receptor solution before analysis.

### In Vitro Permeation Test Method:

Apparatus: Franz diffusion cells—20 mL; Sampling time points: Predose, 1 h, 22 h and 24 h; Sample aliquot: 0.300 mL at each sampling time point; Heating circulator bath temperature: 40° C.±1.0° C.; Temperature on membrane surface: 32° C.±1.0° C.; Skin: Human cadaver skin (Dermatomed); Receptor solution: 0.025%±0.001% brij-98 in 10 mM Phosphate buffered saline (w/v); Extraction solution: Methanol (for surface washing and extraction of analyte from dermis and epidermis); receptor solution (for extraction of analyte from stratum corneum).

### HPLC Method:

Column: Synergy 4μ Max-RP 80A (150×4.6 mm, 4μ); Mobile Phase: Buffer-1 (10 mM ammonium bicarbonate):Acetonitrile:Methanol; 35:35:30, v/v; Flow Rate: 1.000 mL/min; Column Oven Temperature: 40° C.±1.0° C.; Sample Cooler Temperature: 10° C.±1.0° C.; Injection Volume: 50 μL; UV Detection: 240 nm; RT: Halobetasol Propionate: 8.0 to 10.0 min; Rinsing solution: Water:Methanol:Acetonitrile, 60:20:20 v/v; Total Run Time: 16.0 min.

The results for in-vitro permeation studies are summarized in Table 6A and 6B

TABLE 6A

| | | *Analyte | *Analyte | | |
|---|---|---|---|---|---|
| | | unabsorbed on | unabsorbed on | Analyte | Analyte |
| | | skin through | skin through | in stratum | in stratum |
| Skin | Formu- | surface wash | surface wash | corneum | corneum |
| donor | lation | (%) | (ng) | (%) | (ng) |
| 1 | Spray | *18.99 | 9496.67 | 0.20 | 98.70 |
| | Cream | 99.42 | 47088.27 | 0.00 | 0.00 |
| 2 | Spray | *38.10 | 19051.13 | 0.61 | 305.93 |
| | Cream | 106.51 | 50444.00 | 0.00 | 0.00 |

*For spray formulation unabsorbed amount of analyte on skin is the sum of recoveries obtained from surface wash and parafilm samples

TABLE 6B

| | | Analyte | Analyte | Analyte | Analyte |
|---|---|---|---|---|---|
| | | absorbed in | absorbed in | absorbed in | absorbed in |
| Skin | Formu- | epidermis | epidermis | dermis | dermis |
| donor | lation | (%) | (ng) | (%) | (ng) |
| 1 | Spray | 3.86 | 1932.40 | 36.02 | 18011.23 |
| | Cream | 0.23 | 107.53 | 0.70 | 332.10 |
| 2 | Spray | 6.99 | 3495.33 | 29.23 | 14616.00 |
| | Cream | 1.15 | 545.77 | 1.42 | 671.33 |

We claim:

1. A halobetasol topical spray system comprising:
(i) a composition comprising halobetasol;
(ii) a dispensing system comprising:
(a) a pressurized container comprising the composition and
(b) a metered valve assembly comprising a metered valve having a stem connected to an actuator,
wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume angle ranging from about 5° to about 40°.

2. The halobetasol topical spray system of claim 1, wherein the actuator has a diameter ranging from about 5.0 mm to about 20.0 mm, and has a height ranging from about 5.0 mm to about 20.0 mm.

3. The halobetasol topical spray system of claim 1, wherein the metered valve assembly further comprises a mounting cup attached to the pressurized container by crimping, a housing, a spring, a dip tube, a gasket, and wherein the dip tube length ranges from about 50 mm to about 150 mm.

4. The halobetasol topical spray system of claim 1, wherein the dispensing system upon each actuation delivers an average shot weight ranging from about 50 mg to about 150 mg.

5. A halobetasol topical spray system comprising:
(i) a composition comprising halobetasol;
(ii) a dispensing system comprising:
(a) a pressurized container comprising the composition and
(b) a metered valve assembly comprising a metered valve having a stem connected to an actuator,
wherein the actuator has an insert having an orifice diameter in a range of about 0.2 mm to about 1.5 mm, wherein when the actuator is depressed for activation to expel the composition from the pressurized container, forms a plume of droplets, wherein the plume has a plume width ranging from about 10 mm to about 65 mm.

6. The halobetasol topical spray system of claim 5, wherein the actuator has a diameter ranging from about 5.0 mm to about 20.0 mm, and a height ranging from about 5.0 mm to about 20.0 mm.

7. The halobetasol topical spray system of claim 5, wherein the metered valve assembly further comprises a mounting cup attached to the pressurized container by crimping, a housing, a spring, a dip tube, a gasket, and wherein the dip tube length ranges from about 50 mm to about 150 mm.

8. The halobetasol topical spray system of claim 5, wherein the dispensing system upon each actuation delivers an average shot weight ranging from about 50 mg to about 150 mg.

9. A halobetasol topical spray system comprising:
(i) a composition comprising halobetasol;
(ii) a dispensing system comprising:
(a) a pressurized container comprising the composition and
(b) a metered valve assembly comprising a metered valve having a stem connected to an actuator,
wherein the actuator has an insert having an orifice diameter in a range of about 0.2 mm to about 1.5 mm, wherein when the actuator is depressed for activation to expel the composition from the pressurized container forms of droplets with a spray pattern characterized by having one or more features of: a Dₘₐₓ in a range of about 4 to about 100 mm, a Dₘᵢₙ in a range of about 4 mm to about 100 mm, and an ovality of about 1.00 to about 3.00.

10. The halobetasol topical spray system of claim 9, wherein the actuator has a diameter ranging from about 5.0 mm to about 20.0 mm, and wherein the actuator is having a height ranging from about 5.0 mm to about 20.0 mm.

11. The halobetasol topical spray system of claim 9, wherein the metered valve assembly is further comprising a mounting cup attached to the pressurized container by crimping, a housing, a spring, a dip tube, a gasket, and wherein the dip tube length ranges from about 50 mm to about 150 mm.

12. The halobetasol topical spray system of claim 9, wherein the dispensing system upon each actuation delivers an average shot weight ranging from about 50 mg to about 150 mg.

13. A halobetasol topical spray system of claim 9, wherein the droplets form a spray pattern with an area of about 300 mm² to about 900 mm².
